(19) United States
(12) Patent Application Publication
(10) Pub. No.: US 2018/0042553 A1
(43) Pub. Date: Feb. 15, 2018

(54) Implantable Device with a Tail Extension Including Embedded Sensor and Antenna

(71) Applicant: PACESETTER, INC., Sylmar, CA (US)

(72) Inventors: Xiaoyi Min, Camarillo, CA (US); Alex Soriano, Ventura, CA (US); Wisit Lim, Santa Clarita, CA (US); John W. Poore, South Pasadena, CA (US); Tejpal Singh, Stevenson Ranch, CA (US); Perry Li, Temple City, CA (US); Gene A. Bornzin, Simi Valley, CA (US)

(21) Appl. No.: 15/233,772

(22) Filed: Aug. 10, 2016

(51) Int. Cl. A61B 5/00; A61B 5/07; A61B 5/042

(52) U.S. Cl. CPC A61B 5/686; A61B 5/0031; A61B 5/042; A61B 5/076; A61B 5/0006; A61B 2562/16

(57) ABSTRACT

A device and method for an implantable cardiac monitor device are provided comprising a device housing having sensing circuits and radio frequency (RF) communications circuits housed within the housing. The device further comprising a tail extension having a proximal end, a distal end, and an extension body extended there between wherein the proximal end is coupled to the housing. The extension body being formed of a flexible material and including at least one conductor that includes a proximal end conductively coupled to the sensing and RF communications circuits. At least a portion of the conductor of the tail extension forms an antenna to be utilized by the RF communications circuit to communicate to an external device. Further, an electrode is provided on the tail extension and is conductively coupled to the conductor and the sensing circuit.

### BACKGROUND OF THE INVENTION

Embodiments herein generally relate to implantable cardiac monitoring devices.

An implantable cardiac monitoring (ICM) device is a medical device that is implanted in a patient to, among other things, monitor electrical activity of a heart. An ICM device may record cardiac activity of a patient over time and report such cardiac activity to an external device. The ICM device may optionally perform various levels of sophisticated analysis of the cardiac activity and based thereon perform additional recording operations. The ICM device may also be configured to deliver appropriate electrical and/or drug therapy, and as such is also referred to as an implantable medical device (IMD). Examples of IMDs include pacemakers, cardioverters, cardiac rhythm management devices, defibrillators, and the like. The electrical therapy produced by an IMD may include, for example, pacing pulses, cardioverting pulses, and/or defibrillator pulses. The device is used to both provide treatment for the patient and to inform the patient and medical personnel of the physiologic condition of the patient and the status of the treatment.

In general, an ICM includes a battery, memory and electronic circuitry that are hermetically sealed within a metal housing (generally referred to as the “can”). The metal housing typically is formed of titanium and includes a shell with an interconnect cavity, in which the memory, pulse generator and/or processor module reside. The device housing is configured to receive a header assembly. The header assembly comprises a mechanical structure which houses an antenna and a sensing electrode. A feed-through assembly is located at the header receptacle area and is sealed to the device housing to form an interface for conductors to enter/exit the interconnect cavity.

However, ICM devices have experienced some limitations. Certain types of ICM devices include one or more sensing electrodes and an antenna that are located within the ICM device. For example, the sensing electrode/electrodes and antenna may be located in the header of the ICM device. Heretofore, the header assembly structure joined to the device limits the length of the antenna and limits the placement of the sensing electrode/electrodes relative to the region of interest.

A need remains for improved ICM devices and methods of manufacture thereof.

### SUMMARY

In accordance with embodiments herein, a device is provided for an implantable cardiac monitor device comprising a device housing having a sensing circuit and a radio frequency (RF) communications circuit housed within the housing. The device further comprising a tail extension having a proximal end, a distal end, and an extension body extended there between wherein the proximal end is coupled to the housing. The extension body is formed of a flexible material and includes at least one conductor that includes a proximal end conductively coupled to the sensing and RF communications circuit. At least a portion of the conductor of the tail extension forms an antenna to be utilized by the RF communications circuit to communicate with an external device. Further, an electrode is provided on the tail extension and is conductively coupled to the conductor and the sensing circuit.

Optionally, the portion of the conductor that forms the antenna is electrically coupled to the electrode. In at least one embodiment, the conductor consists of a single conductor that both forms the antenna and carries cardiac sensed signals from the electrode. In at least one other embodiment, the conductor may include first and second conductors, wherein the first conductor is coupled to the electrode and is configured to carry cardiac sensed signals, and the second conductor forms the antenna and is configured to carry communications data to and/or from the RF communications circuit.

Optionally, the proximal end of the tail extension is joined directly at a non-header interface on a surface of the device housing. The device further comprises a feed-through assembly joined to the device housing. The feed-through assembly including a single conductor extending there through. The single conductor has a proximal end connected to the sensing and RF communications circuits and a distal end projecting from the feed-through assembly.

Optionally, the device housing further comprises a filter circuit conductively coupled between the at least one conductor and the sensing and RF communications circuits. The filter circuit is configured to block RF transmissions from reaching the sensing circuit. The filter circuit includes an inductive element and a capacitive element connected in series with one another at an intermediate node. The sensing circuit joins to the intermediate node. The capacitor element is configured to form an open circuit when experiencing cardiac sensing signals and to form a closed circuit when experiencing RF transmissions. The filter further comprises a low pass filter branch configured to pass cardiac sensed signals and a band pass filter branch configured to pass RF communications in a frequency range that includes approximately 2.4 GHz.

Optionally, the tail extension of the device comprises a predetermined length between the distal end and the proximal end. The predetermined length is tuned based on the center of frequency of the RF communications bandwidth.

In accordance with embodiments herein, a method is provided for implanting a cardiac monitor device comprising positioning a device subcutaneously, the device comprising a device housing having a sensing circuit and a radio frequency (RF) communications circuit housed within the housing. The device further comprises a tail extension having an extension body with a proximal end joined to the device housing. The extension body is formed of a flexible material and includes at least one conductor. The tail extension is positioned in a subcutaneous area with an electrode provided on the tail extension located proximate to a region of interest (ROI) in a heart. The electrode collects cardiac signals from the ROI provided on the tail extension and conveys the RF communications data to an external device using an antenna that is formed from at least a portion of the conductor.

Optionally, the method comprises a positioning operation of locating a distal end of the tail extension remote from the device housing and an electrode provided at the distal end of the tail extension located proximate to a region of interest in the heart. The conductor includes a first and second conductor, wherein the first conductor is coupled to the electrode and configured to carry the cardiac signals and the second conductor forms the antenna and is configured to carry the RF communications data to and/or from the RF communications circuit.

Optionally, the method comprises filtering the cardiac signals to isolate the RF communications circuit from the cardiac signals sensed over the conductor, and filtering the RF communications to isolate the sensing circuit from the RF communications data carried by the conductor. The collecting and conveying operations utilize a common conductor in the tail extension. The filtering operation comprises low pass filtering to pass the cardiac signals along a sensing branch and band pass filtering to pass RF communications data that is in a frequency range that includes approximately 2.4 GHs, along a communications branch.

Optionally, the method comprises inserting the tail extension into a lumen in a tail implant tool and inserting the tail implant tool subcutaneously to a location proximate to the ROI. The tail implant tool is removed while leaving the tail extension implanted. Optionally, the ROI is located proximate to an atrium of the heart such that the cardiac signals include sensed P-waves.

In accordance with embodiments herein, a method is provided for providing an implantable cardiac monitor device comprising providing a device comprising a device housing have a sensing circuit and a radio frequency (RF) communications circuit housed within the housing. The method further comprising joining a tail extension to the device housing, the tail extension having an extension body with a proximal end joined to the device housing. The extension body formed of a flexible material and including at least one conductor. An electrode may be positioned on the tail extension to sense cardiac signals when the tail extension is located proximate to a region of interest (ROI) in the heart. A portion of the conductor forms an antenna and tuning the antenna and RF communications circuit to convey RF communications data to an external device utilizing a predetermined communications frequency.

Optionally, the tuning operation includes tuning the antenna to utilize the predetermined communications frequency centered at one of 2.4 GHz and 400 MHz.

### BRIEF DESCRIPTION OF THE DRAWINGS

FIG. 1 illustrates an implantable cardiac monitoring (ICM) device intended for subcutaneous implantation at a site near the heart in accordance with embodiments herein.

FIG. 2 illustrates a side perspective view of the ICM device in accordance with embodiments herein.

FIG. 3 illustrates a side sectional view of a tail extension joined to the ICM device in accordance with embodiments herein.

FIG. 4 illustrates a side sectional view of a device housing in accordance with embodiments herein.

FIG. 5A illustrates a side sectional view of the tail extension joined to the device housing in accordance with embodiments herein.

FIG. 5B. illustrates a side sectional view of the tail extension joined to the device housing in accordance with embodiments herein.

FIG. 6A illustrates a side sectional view of a tail extension separated from a device housing in accordance with embodiments herein.

FIG. 6B illustrates a side sectional view of a tail extension joined to the device housing in accordance with embodiments herein.

FIG. 6C illustrates a flowchart method for implanting and connecting a tail extension and a device housing in accordance with embodiments herein.

FIG. 7 illustrates a block diagram of an exemplary ICM device that is configured to be implanted into the patient in accordance with embodiments herein.

FIG. 8 illustrates a circuit diagram of the exemplary ICM device of FIG. 7 in accordance with embodiments herein.

FIG. 9 illustrates a block diagram of an alternative exemplary ICM device that is configured to be implanted into the patient in accordance with embodiments herein.

### DETAILED DESCRIPTION

In accordance with the embodiments herein, devices and methods are described that afford improved placement of a sensing electrode and antenna performance in implantable cardiac monitoring (ICM) devices (including implantable medical devices configured to deliver therapies). In accordance with at least some embodiments, the devices and methods described herein provide improved sensing of cardiac signals as compared to conventional implantable cardiac monitoring devices and eliminate conventional structural limitations. In accordance with at least some embodiments, the devices and methods described herein provide improved wireless communications performance in a compact footprint.

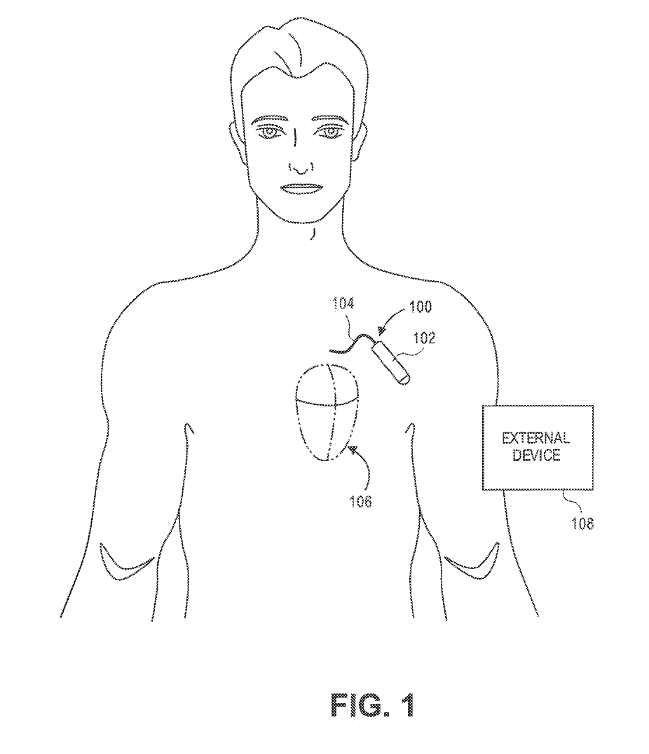

FIG. 1 illustrates an implantable cardiac monitoring (ICM) device 100 intended for subcutaneous implantation in a patient at a site near a heart 106. The ICM device 100 includes a device housing 102 that is joined to a proximal end of a tail extension 104. The device housing 102 is a solid component that communicates with an external device 108. The tail extension 104 is a flexible component that receives cardiac signals from the heart 106. The ICM device 100 is placed in a subcutaneous area of the patient with a distal end of the tail extension 104 located proximate to a region of interest (ROI) in the heart. For example, the ROI is located proximate to an atrium of the heart such that cardiac signals that are collected by the ICM device 100 include P-waves. Optionally, the ROI could be a number of anatomical aspects of a patient, such as the heart wall, the right ventricle, left ventricle, etc. The ICM device 100 may be positioned in various orientations relative to a patient anatomy, such as relative to various aspects of the heart, parallel to the sternum, non-parallel to the sternum, and the like.

The device housing 102 includes various other components such as one or more sense circuits for receiving and collecting cardiac signals from one or more combinations of electrodes, a microprocessor for processing the cardiac signals in accordance with various algorithms (e.g., an AF detection algorithm), a memory for temporary storage of electrogram data, a device memory for long-term storage of electrogram data (e.g., based on certain triggering events, such as AF detection), sensors for detecting patient activity and a battery for powering circuits and other components.

The ICM device 100 senses near field and/or far field cardiac signals and stores the cardiac signals as electrogram data. The ICM device 100 processes the electrogram (EGM) data in various manners such as to detect physiologic characteristics of interest (e.g., arrhythmias). The ICM device 100 automatically records segments of the electrogram data in temporary or long-term memory based on identification of the characteristics of interest. By way of example, the EGM data may be stored in memory of the ICM device 100 until subsequent transmission to the external device 108. Electrogram processing and arrhythmia detection is provided for, at least in part, by algorithms embodied in program instructions that are executed by one or more microprocessors. In one configuration, the monitoring device is operative to detect atrial fibrillation.

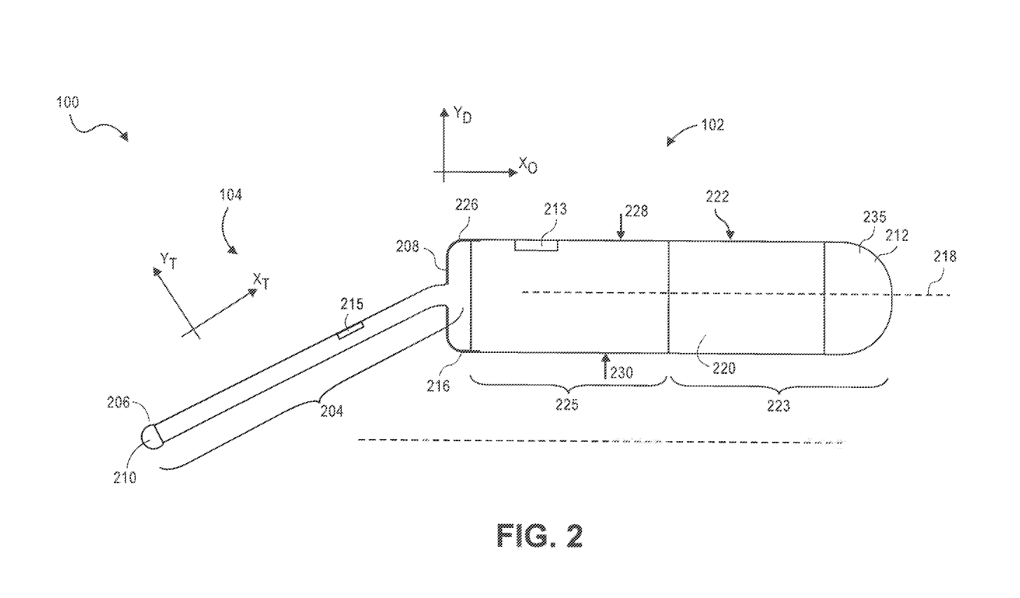

FIG. 2 illustrates a side perspective view of the ICM device 100 in accordance with an embodiment herein. FIG. 2 illustrates the tail extension 104 and device housing 102 of the ICM device 100 in more detail.

The tail extension 104 is elongated to extend along a longitudinal axis (designated by arrow X_{T}). The tail extension 104 is formed in a tubular shape about the longitudinal axis (designated by the arrow X_{T}) and is generally circular in shape about a cross sectional axis (designated by arrow Y_{T}). For example, the tail extension 104 is shaped similarly to a hollow wire. Optionally, the tail extension could be formed with a number of shapes. Additionally, the tail extension 104 is a flexible structure made of a flexible, insulating, biocompatible material (e.g., silicone rubber, polyurethane, etc.).

The tail extension 104 comprises a distal end 206 and a proximal end 208, with an extension body 204 spanning between the distal end 206 and the proximal end 208. The distal end 206 of the tail extension 104 is located remote from the device housing 102. The proximal end 208 of the tail extension 104 is positioned near, and coupled to, the device housing 102.

One or more primary sensing electrodes 210 are positioned at the distal end 206 of the tail extension 104. One or more secondary sensing electrodes 212, 213, 215 may be provided on the device housing 102 and/or on the tail extension 104 at an intermediate point(s) along the length of the extension body 204. The sensing electrodes 210, 212, 213, 215 are configured to receive cardiac signals from the heart 106. The sensing electrodes 210, 212, 213, 215 may vary in shape and size.

The device housing 102 extends along a longitudinal axis 218. In the embodiment of FIG. 2, the device housing 102 is formed in a rectangular shape about the longitudinal axis 218 and in a rectangular shape about a cross sectional axis (designated by the arrow Y_{D}). However, the device housing 102 may be constructed with alternative shapes (e.g., a tubular shape, a circular shape, a shape similar to pacemakers, ICDs, CRM devices and other implantable devices). The device housing 102 comprises opposed elongated sides 220, 222, a top edge 228, a bottom edge 230, a header end 226 and a base end 235. The base end 235 includes the secondary electrode 212 provided thereon. The opposed elongated sides 220, 222 are generally parallel, but may be constructed with alternative contours. The opposed sides 220, 222 merge with the top and bottom edges 228 and 230 along smooth beveled regions about the cross sectional axis (designated by the arrow Y_{D}).

In FIG. 2, the device housing 102 is constructed in two pieces, namely with a battery portion 223 and an electronics shell portion 225. The battery portion 223 is formed as a self-contained, hermetically sealed battery. The electronics shell portion 225 encloses the various electronic components and circuits as discussed herein.

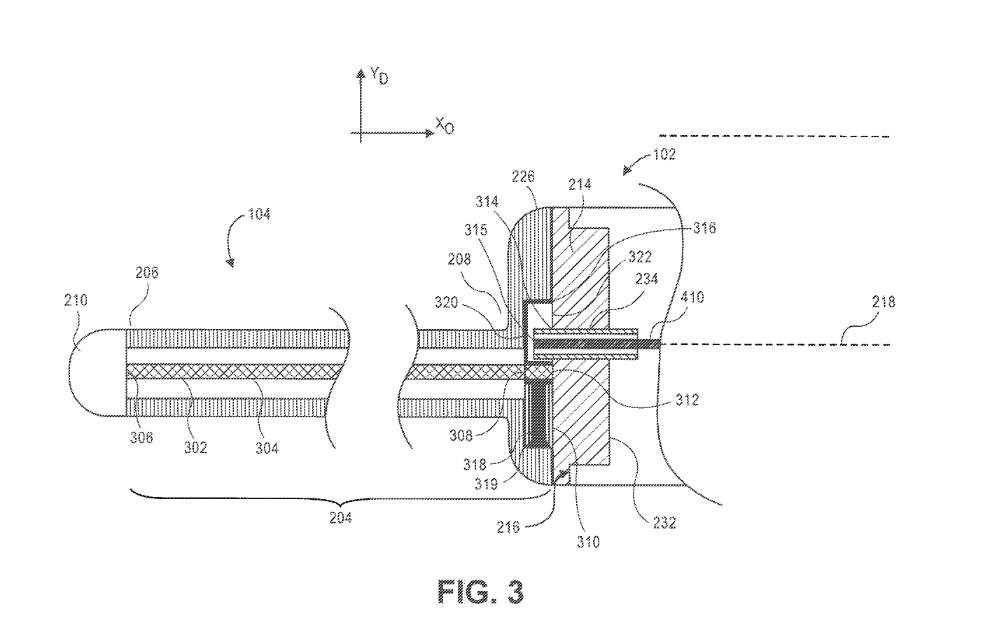

FIG. 3 illustrates a detailed cross-sectional view of the tail extension 104 joined to the device housing 102 in accordance with an embodiment. The tail extension 104 and the device housing 102 are oriented to extend along the longitudinal axis 218. FIG. 3 illustrates the tail extension 104 and header end 226 of the device housing 102 in more detail.

The tail extension 104 comprises at least one conductor 302. The conductor 302 is positioned generally centered within the tail extension 104 and extends longitudinally the length of the extension body 204. The conductor 302 may be a single strand wire, a multi-strand wire, a filar, or the like. Optionally, the conductor 302 may be formed as a tubular mesh. The tubular mesh may be constructed to include an interior lumen that is configured to receive an insertion tool (e.g., a guide wire, stylet, etc.).

The conductor 302 includes a distal end 306 and a proximal end 308. The distal end 306 of the conductor is conductively joined to the sensing electrode 210. The proximal end 308 of the conductor is positioned proximate to the device housing 102.

In accordance with embodiments herein, all or a portion of the conductor 302 within the tail extension 104 is also utilized as an antenna 304. The antenna 304, for example, may be utilized to support communications in accordance with various wireless protocols such as, but not limited to, a Medical Implant Communications Service (MICS) protocol, a Bluetooth protocol, a Bluetooth Low Energy protocol, a Wi-Fi protocol, and the like.

The tail extension 104 (and antenna 304) may be constructed with various lengths based upon select criteria. For example, the length of the tail extension 104 may be varied based upon the application for which the ICM device 100 is constructed. For example, different length tail extensions may be used when monitoring P-waves, or monitoring R-waves. Additionally or alternatively, one or more length(s) of tail extensions may be useful when sensing electrical activity of the heart as R-waves or P-waves, whereas one or more other length(s) of tail extensions may be useful when monitoring impedance, such as across various blood pools in the heart and/or in vessels proximate to the heart (e.g., the five great vessels of the heart). In addition, the length of the tail extension may be varied based on a desired combination of anatomical locations to be monitored by the ICM device 100. For example, it may be desirable to locate the device housing 102 on one side of the heart (e.g., proximate the apex of the heart), while positioning the distal end of the tail extension 104 proximate to a different side/portion of the heart (e.g., proximate to the base of the heart). The length of the tail extension may be varied for other reasons as well.

In addition, the length of the tail extension may also be based on characteristics of the antenna 304. In particular, the length of the antenna is set to a predetermined or select length based on various criteria. For example, the length of the conductor, that defines the antenna 304, may be set to a predetermined or select length in order to electrically tune the antenna 304 and the RF communications circuit to a predetermined frequency. For example, the length of the antenna may be set to a predetermined length in order to provide a desired antenna performance when communicating RF communications data centered at a select center frequency (e.g., 2.4 GHz, 400 MHz, etc.). Optionally, the length of the antenna may be sized to a different length to communicate over a different frequency.

Optionally, more than one conductor may be provided within the tail extension with the conductors conductively isolated from one another (e.g., individually insulated). When more than one conductively insulated conductor is provided within the tail extension, a first conductor (sensor conductor) may be dedicated to carrying sensed signals from the sensing electrode 210 on the distal end, while a second conductor (antenna conductor) may be dedicated to transmitting and receiving communications data. When separate sensor and antenna conductors are provided in the tail extension, the sensor and antenna conductors may be shaped and formed to provide a desired level of performance relative to the corresponding usage. For example, a sensor conductor may be sized differently from an antenna conductor. Additionally or alternatively, the sensor conductor may be formed from a different material than the antenna conductor. Additionally or alternatively, the sensor conductor may utilize a multi-strand filar with a select first diameter, while the antenna conductor is a single strand with a different (e.g., thicker) diameter.

Optionally, when separate sensor and antenna conductors are utilized, the antenna conductor may be formed with a different length than the sensor conductor. For example, in some applications, it may be desirable for the antenna conductor to have a length that is a fraction of a wavelength of the center communications frequency (e.g., one quarter wavelength in length). In addition, it may be desirable for the overall length of the tail extension to be longer that the predetermined length for the antenna conductor. In the foregoing example, the sensor conductor may extend the full length of the tail extension X (corresponding to a sensor spacing or sensor placement parameter), while the antenna conductor may extend a shorter length Y (corresponding to a timing related parameter). Utilizing separate conductors for sensing and for the antenna provides more flexibility to tailor the design parameters of the sensing characteristics and the antenna characteristics.

In FIG. 3, the header end 226 of the device housing 102 comprises a housing mounting surface 216. The housing mounting surface 216 is a non-header interface that comprises a recessed chamber 232. The recessed chamber 232 is positioned to open on to the housing mounting surface 216. The recessed chamber 232 is shaped and dimensioned to receive a feed-through assembly 214 on the device housing 102. In the present example, the recessed chamber 232 is rectangular in shape, although alternative shapes may be used based on the shape of the feed-through assembly 214. A passage 234 extends from the recessed chamber 232 through the feed-through assembly 214 along the longitudinal axis 218.

The size and/or shape of the proximal end 208 of the tail extension 104 corresponds to the size and/or shape of the header end 226 of the device housing 102. For example, the proximal end 208 of the tail extension 104 may be formed in a hollow-rectangular shape to form a cap over the device housing 102 at the header end 226 when joined. Optionally, alternative shapes may be used based on the shape and/or size of the housing mounting surface 216 of the device housing 102. The hollow-rectangular shape of the proximal end 208 of the tail extension may be joined to the device housing by a press fit onto the device housing 102. Additionally or alternatively, the proximal end 208 of the tail extension may be joined to the device housing by alternative methods.

In FIG. 3, a collar 310 is joined to the feed-through assembly 214 of the device housing 102 along the longitudinal axis 218. The collar 310 comprises a distal end 320 and a proximal end 322. The distal end 320 is positioned facing the sensing electrode 210 of the tail extension 104. The proximal end 322 of the collar joins to the feed-through assembly 214. The collar 310 may be joined to the feed-through assembly by various methods. For example, the collar 310 may be joined to the feed-through assembly by welding, over-molding, mechanical fasteners, or the like. For example, the collar 310 may be welded to the feed-through assembly 214 at a weld joint 316.

Furthermore, the collar 310 comprises one or more openings. The distal end 320 of the collar 310 comprises a distal-opening 312. The distal-opening 312 is sized and shaped to receive the proximal end 308 of the conductor 302 of the tail extension 104. The proximal end 322 of the collar 310 comprises a proximal-opening 314. The proximal-opening 314 is sized and shaped to receive the passage 234 of the feed-through assembly 214.

According to an embodiment, the collar 310 is configured with a third opening 318. The third opening 318 may be positioned perpendicular to the longitudinal axis 218 and may be constructed to intersect with the distal-opening 312. The third opening 318 comprises a set-screw 319. The set-screw 319 of the third opening 318 joins the proximal end 308 of the conductor 302 to the collar 310 to electrically couple the conductor 302 with the collar 310.

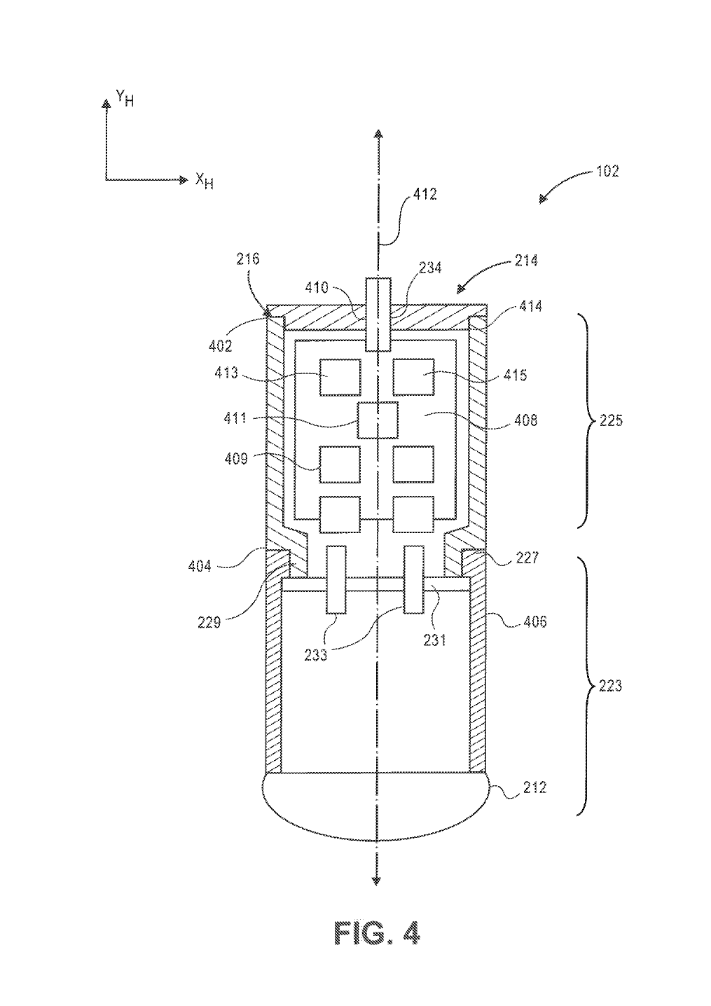

FIG. 4 illustrates a detailed cross-sectional view of the device housing 102 formed in accordance with an embodiment. The device housing 102 is oriented to extend along the longitudinal axis 218. FIG. 4 illustrates the battery portion 223 and electronics shell portion 225 in more detail. The distal end of the battery portion 223 includes the secondary electrode 212, while a proximal end 227 includes a shell reception opening sized and shaped to receive an end 229 of the electronics shell portion 225. A battery feed-through 231 is provided at the interface between the electronics shell portion 225 and battery portion 223. Conductors 233 extend through the battery feed-through 231 to provide electrical connections to a battery 406. A weld joint 404 hermetically seals the electronics shell portion 225 to the battery portion 223.

FIG. 4 further illustrates the interface between the feed-through assembly 214 and the electronics shell portion 225 at the housing mounting surface 216. A weld joint 402 hermetically seals the feed-through assembly 214 to the proximal end of the electronics shell portion 225. In the foregoing example, welds are provided at component interfaces, however it is understood that alternative attachment techniques may be utilized.

The electronics shell portion 225 includes various circuits, components and memory based on the individual operations and application for which the ICM device 100 is configured. In FIG. 4, an example electronics board 408 includes one or more processors 409, memory 411, sensing circuit 413, and RF communications circuit 415, as well as other electrical components. Examples of the various electronic components may include sensing circuitry to sense cardiac signals of interest, one or more processors to perform monitoring operations, transceiver circuitry to communicate with external devices and other components as described herein.

The sensing circuit 413 and RF communications circuit 415 are electrically coupled with a single conductor 410 at a proximal end 414. A distal end 412 of the single conductor 410 extends from the electronics board 408 through the passage 234 of the feed-through assembly 214. For example, in the present example, the single conductor 410 is a single-strand wire that is electrically coupled to the electronic components on the electronics board 408 (e.g. the sensing circuit 413 and RF communications circuit 415). The single conductor 410 projects outward from the feed-through assembly 214 to provide a connection point to be conductively joined to the proximal end of one or more conductors within the tail extension 104.

Returning to FIG. 3, the single conductor 410, extending from the electronics shell portion 225 of the device housing 102, may be received into the proximal-opening 314 of the collar 310. The single conductor 410 may be conductively coupled to the collar 310. For example, the single conductor 410 may be conductively coupled to the collar 310 by a weld joint 315. Additionally or alternatively, the single conductor may be joined to the collar using alternative attachment techniques. The collar 310 interconnects the proximal end 308 of the conductor 302 of the tail extension 104 with the single conductor 410 extending from the device housing 102. For example, the collar 310 conductively couples the proximal end 308 of the conductor 302 with the sensing circuit 413 and RF communications circuit 415. The collar 310 enables electrical communication between the sensing electrode 210 of the tail extension 104 and the electronic components of the device housing 102.

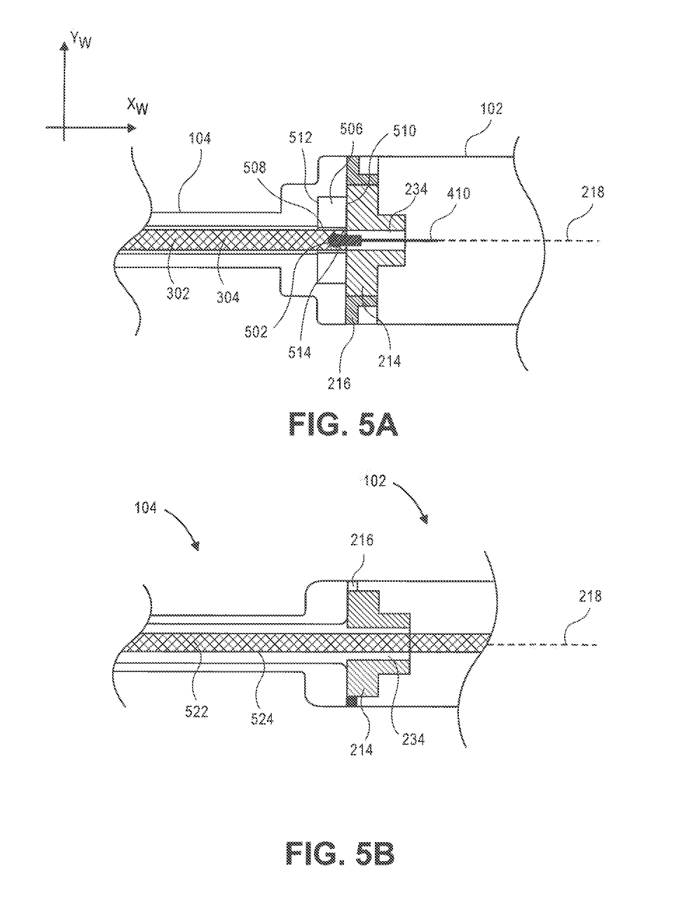

FIG. 5A illustrates an alternative example of a connection mechanism for interconnecting the conductor 302 of the tail extension 104 to the electronic components of the device housing 102. A collar 506 is joined to the feed-through assembly 214. A distal end 512 of the collar 506 is positioned facing away from the feed-through assembly 214. A proximal end 510 of the collar is positioned facing towards the feed-through assembly 214. In the embodiment of FIG. 5A, the collar 506 comprises a collar-passage 508. The collar-passage 508 is open along the longitudinal axis 218. The collar-passage 508 is sized and shaped to receive the conductor 302 of the tail extension 104 on the distal end 512 and to receive the passage 234 of the feed-through assembly 214 on the proximal end 510.

The single conductor 410 is electrically coupled to the electronic components of the device housing 102 at the proximal end 414 (not shown). The distal end 412 of the single conductor 410 extends from the device housing through the feed-through assembly 214 along the longitudinal axis 218. The distal end 412 of the single conductor 410 is conductively joined to a pin 514. The pin 514 extends from the distal end 412 of the single conductor 410 towards the collar 506 along the longitudinal axis 218. The conductor 302 of the tail extension 104 may be conductively coupled with the pin 514 at a weld joint 502. For example, the pin 514 conductively couples the proximal end 308 of the conductor 302 with the sensing circuit 413 and RF communications circuit 415.

FIG. 5B illustrates an alternative example of a connection mechanism for interconnecting the conductor of the tail extension 104 to the electronic components of the device housing 102. In accordance with an embodiment in FIG. 5B, a single conductor 522 (corresponding to the conductor 302 of FIG. 3) extends within the tail extension 104 along the longitudinal axis 218. All or a portion of the conductor 522 within the tail extension 104 is also utilized as an antenna 524 (corresponding to the antenna 304 of FIG. 3). The single conductor 522 is conductively joined to the sensing electrode 210 on the distal end of the tail extension 104 (not shown). The single conductor 522 extends between the sensing electrode 210 and the device housing 102. In the present embodiment, the single conductor 522 extends through the passage 234 of the feed-through assembly 214 and into the device housing 102. The single conductor 522 is electrically coupled to the electronic components of the device housing 102 (not shown). For example, the single conductor 522 is conductively joined to the sensing electrode 210 at a distal end, and conductively joined to the electronic components of the device housing 102 at a proximal end.

The previous embodiments illustrate three examples demonstrating how the conductor of the tail extension 104 may be conductively coupled to the electronic components of the device housing 102. However, it is understood that alternative attachment techniques may be utilized. The conductor of the tail extension 104, electrically coupled with the electronic components of the device housing 102, enables electrical communication between the sensing electrode 210 of the tail extension and the electronic components of the device housing 102. The conductor 302, electrically coupled to the sensing electrode 210, performs the operations of conveying collected cardiac sensing signals from the ROI in the heart to the sensing circuitry 413 of the device housing. The portion of the conductor 302 that forms the antenna 304 performs the operations of communicating the RF communications data to and/or from the RF communications circuitry 415 of the device housing 102 and the external device 108.

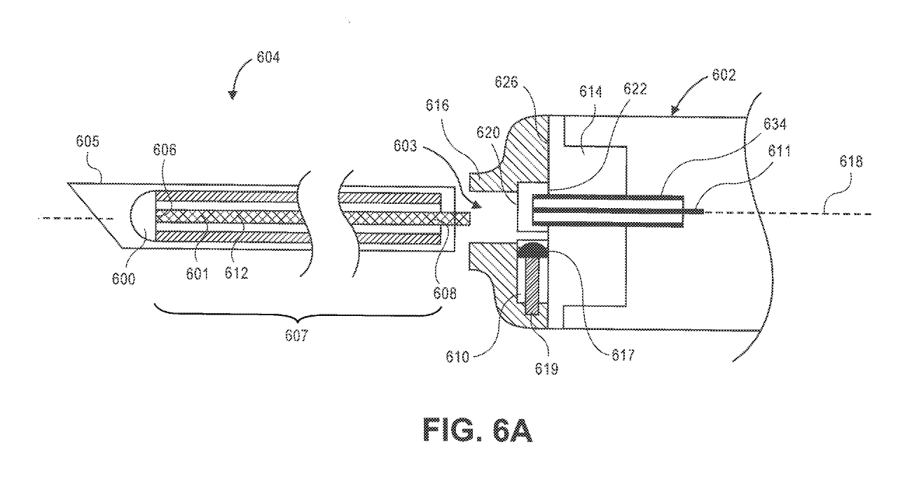

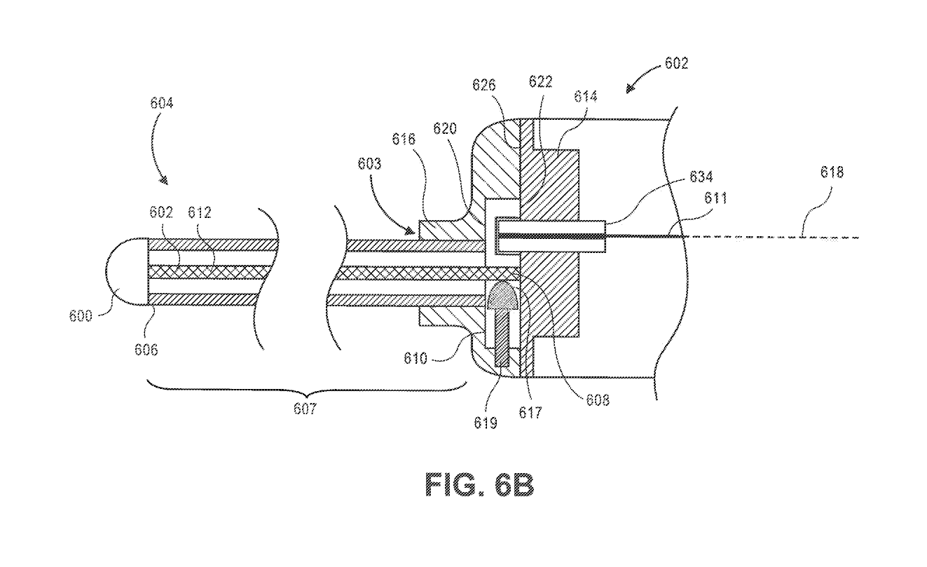

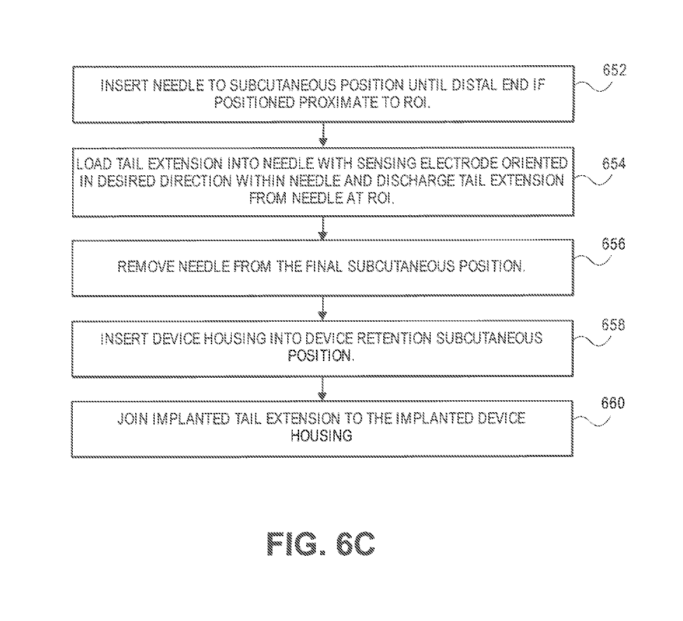

FIGS. 6A and 6B illustrate an embodiment of a tail extension 604 and a device housing 602 formed in accordance with an alternative embodiment. The tail extension 604 and device housing 602 are configured to be subcutaneously positioned at a ROI of the heart prior to the tail extension 604 being joined to the device housing 602. FIG. 6A illustrates the tail extension 604 separated from the device housing 602. FIG. 6B illustrates the tail extension 604 joined to the device housing 602. FIGS. 6A and 6B will be discussed in detail together in connection with FIG. 6C.

FIG. 6C illustrates a flowchart for a process for separately subcutaneously implanting the tail extension 604 and the device housing 602, and then joining the tail extension 604 to the device housing 602 after implant.

The tail extension 604 (corresponding to the tail extension 104 of FIG. 2) comprises a distal end 606 and a proximal end 608, with an extension body 607 spanning between the distal end 606 and the proximal end 608. The distal end 606 of the tail extension 604 is located remote from the device housing 602. A primary sensing electrode 600 (corresponding to the primary sensing electrode 210 of FIG. 2) is positioned at the distal end 606 of the tail extension 604. The proximal end 608 of the tail extension 604 is positioned near, and configured to be coupled to, the device housing 602.

A single conductor 601 (corresponding to the conductor 302 of FIG. 3) extends within the tail extension 604 along a longitudinal axis 618. For example, the single conductor 601 extends between the sensing electrode 600 and the proximal end 608 of the tail extension 604. All or a portion of the conductor 601 within the tail extension 604 is also utilized as an antenna 612 (corresponding to the antenna 304 of FIG. 3).

A header gasket 616 forms a cap over the device housing 602 at the header end 626. The header gasket 616 is a hollow piece that is sized and/or shaped to fit over the device housing 602. The header gasket 616 is a flexible structure made of a flexible, insulating, biocompatible material (e.g., silicone rubber, polyurethane, etc.). The header gasket 616 comprises a receiving slot 603 that is sized and/or shaped to receive the proximal end 608 of the tail extension 604. For example, the receiving slot 603 of the header gasket 616 receives the proximal end 608 of the tail extension 604 in order to join the tail extension 604 to the device housing 602. Optionally, the header gasket 616 may be omitted entirely.

In FIG. 6C, beginning at 652, a needle 605 is inserted into a subcutaneous position at a ROI of the heart. The needle 605 is maneuvered until a distal end of the needle 605 is located at a final subcutaneous location of interest (not shown). The final subcutaneous location could be a ROI proximate to the heart. For example, the final location may be positioned on one side of the heart (e.g., proximate the base of the heart). Additionally or alternatively, the final location may be any other ROI that is proximate to the heart. Optionally, the final location may be proximate to another anatomy of interest, such as an ROI in or near the lungs, an organ, a portion of the nervous system, the spine, the brainstem, the brain, etc.

At 654, the distal end 606 of the tail extension 604 is loaded into the needle 605. For example, the distal end 606 with the sensing electrode 600 is inserted through the needle 605 and discharged from the distal end of the needle in order to position the sensing electrode 600 at the final subcutaneous location. Optionally, the tail extension 604 may be loaded into the needle before or after the needle is inserted to the ROI. At 656, the needle 605 is removed from the final subcutaneous position leaving the tail extension 604 implanted and the sensing electrode 600 at the final subcutaneous position.

At 658, the device housing 602 is implanted to a device retention subcutaneous position. Various methods and tools may be used to implant the device housing 602. For example, a sheath, catheter and the like may be introduced through a vein or artery to a device retention position. Optionally, the device housing 602 may be located in a pocket through an open incision (e.g., at a sub-clavicle pocket). For example, it may be desirable to position the device housing 602 on a different side/portion of the heart (e.g., proximate the apex of the heart) from the distal end 606 of the tail extension 604. At 660, once the device housing 602 is implanted at the desired location, the implanted tail extension 604 is joined to the implanted device housing 602. The device housing 602 and the tail extension 604 may be secured to one another in various manners. For example, device housing 602 and the tail extension 604 may be secured to one another in a manner similar to attaching a proximal end of a lead to a pacemaker, cardioverter defibrillator, neuro-stimulation device and the like.

FIG. 6B illustrates the tail extension 604 joined to the header gasket 616. In the embodiment of FIG. 6B, a collar 610 (corresponding to the collar 310 of FIG. 3) is joined to a feed-through assembly 614 of the device housing 602 along the longitudinal axis 618. The collar receives the conductor 601 of the tail extension at a distal end 620. The collar receives a single conductor 611 (corresponding to the single conductor 410 of FIG. 3) extending from the device housing 602 through a passage 634 of the feed-through assembly 614 of the device housing 602 at a proximal end 622.

The collar 610 is configured with an opening 617 perpendicular to the longitudinal axis 618. The opening 617 comprises a set-screw 619. The header gasket 616 flexes to allow access to the collar 610 in order to join the conductor 601 of the tail extension 604 to the collar 610 with the set-screw 619. The set-screw 619 of the opening 617 joins the conductor 601 to the collar 610 to electrically couple the conductor 601 with the collar 610. The collar 610 interconnects the conductor 601 of the tail extension 604 with the single conductor 611 extending from the device housing 602. The collar 610 enables electrical communication between the sensing electrode 600 of the tail extension 604 and the electronic components of the device housing 602. Once the conductor 601 is joined to the collar 610, the header gasket 616 rebounds to the original position. In the original position, the proximal end 608 of the tail extension 604 is held to the header gasket 616 by a press fit into the receiving slot 603.

The previous embodiment illustrates one example demonstrating how the conductor of the tail extension 604 may be conductively coupled to the electronic components of the device housing 602 after the tail extension 604 and the device housing 602 have been individually subcutaneously implanted. However, it is understood that alternative attachment techniques may be utilized.

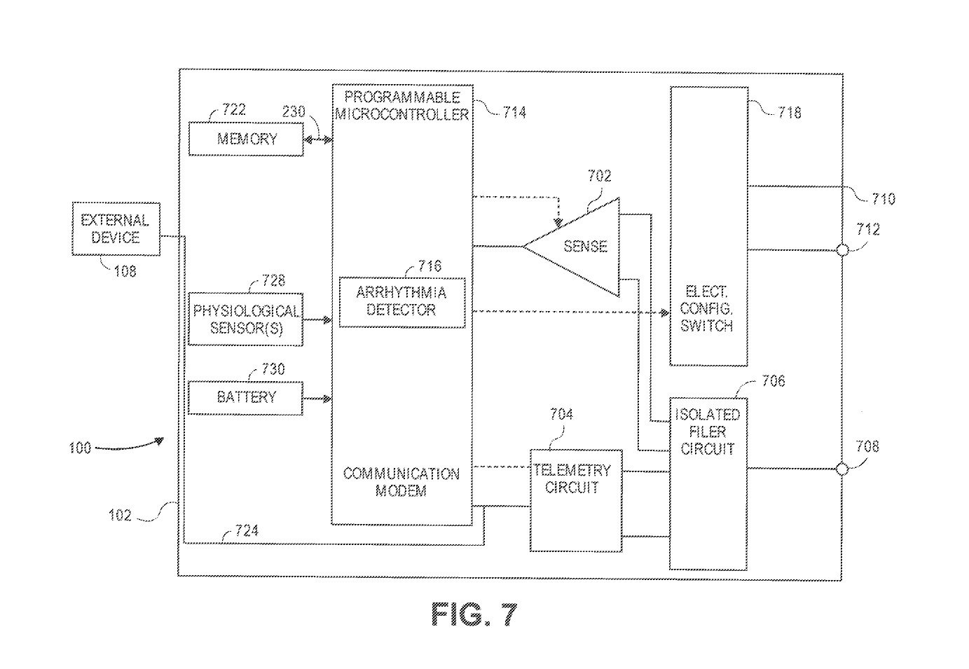

FIG. 7 illustrates a block diagram of the ICM device 100 that is configured to be subcutaneously implanted into the patient. Optionally, the ICM device 100 may be provided as an external device that is worn, held or otherwise located proximate to the patient during operation. The ICM device 100 may be implemented to monitor ventricular activity alone, or both ventricular and atrial activity through sensing circuitry. The ICM device 100 has the device housing 102 to hold the electronic/computing components. The device housing 102 (which is often referred to as the “can”, “case”, “encasing”, or “case electrode”) may be programmably selected to act as an electrode for certain sensing modes.

In the present embodiment, the sensing electrode 210 positioned on the tail extension 104 is coupled to a terminal 708. The device housing 102 further includes a connector (not shown) with at least one terminal 710 and preferably a second terminal 712. The terminals 710, 712 may be coupled to additional sensing electrodes on the device housing, on the tail extension, or located otherwise. For example, the terminals 710, 712 may be coupled to the sensing electrodes 212, 213 and/or 215 of FIG. 2. Additionally or alternatively, the terminals 710, 712 may be connected to one or more leads having one or more electrodes provided thereon, where the electrodes are located in various locations about the heart. The type and location of each electrode may vary.

The ICM device 100 is configured to be placed subcutaneously utilizing a minimally invasive approach. Subcutaneous electrodes are provided on the device housing 102 to simplify the implant procedure and eliminate a need for a transvenous lead system. The sensing electrodes may be located on opposite sides/ends of the device and designed to provide robust episode detection through consistent contact at a sensor-tissue interface. The ICM device 100 may be configured to be activated by the patient or automatically activated, in connection with recording subcutaneous ECG signals.

The ICM device 100 includes a programmable microcontroller 714 that controls various operations of the ICM device 100, including cardiac monitoring. Microcontroller 714 includes a microprocessor (or equivalent control circuitry), RAM and/or ROM memory, logic and timing circuitry, state machine circuitry, and I/O circuitry. The microcontroller 714 also performs the operations in connection with collecting cardiac activity data and analyzing the cardiac activity data to identify episodes of interest. Microcontroller 714 includes an arrhythmia detector 716 that is configured to analyze cardiac activity data to identify potential AF episodes as well as other arrhythmias (e.g. Tachcardias, Bradycardias, Asystole, etc.).

A switch 718 is optionally provided to allow selection of different electrode configurations connected to the terminals 710, 712 under the control of the microcontroller 714. The switch 718 is controlled by a control signal from the microcontroller 714. Optionally, the switch 718 may be omitted and the I/O circuits directly connected to the housing electrode and a second electrode.

The ICM device 100 is further equipped with telemetry circuitry 704. The telemetry circuitry 704 uses high frequency modulation, for example uses RF or Bluetooth telemetry protocols. The telemetry circuitry 704 may include one or more transceivers. For example, the telemetry circuitry 704 may be coupled to the antenna 304 in the tail extension 104 that transmits communications signals in a high frequency range that will travel through the body tissue in fluids without stimulating the heart or being felt by the patient.

The ICM device 100 includes sensing circuitry 702 selectively coupled to one or more electrodes that perform sensing operations to detect cardiac activity data indicative of cardiac activity. For example, the sensing circuitry 702 may be coupled to the conductor 302 in the tail extension that conveys sensing signals from the ROI in the heart in a low frequency range. The sensing circuitry 702 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest.

An isolating filter circuit 706 is electrically coupled between the terminal 708 and the telemetry circuitry 704 and the sensing circuitry 702. The isolating filter circuit 706 receives low frequency cardiac signal data communicated by the conductor 302 and high frequency RF communications data communicated by the antenna 304 through the terminal 708. The isolating filter circuit 706 is provided to pass or block the data communicated over the single-strand conductor 302 and antenna 304. The isolating filter circuit 706 passes the low frequency data to the sensing circuitry 702 and blocks the low frequency data from the telemetry circuitry 704. For example, the isolating filter receives low frequency sensed cardiac signals from the conductor 302 of the tail extension 104. The isolating filter circuit 706 passes the sensed cardiac signals to the sensing circuitry 702. The isolating filter circuit 706 blocks the sensed cardiac signals from the telemetry circuitry 704.

The isolating filter circuit 706 passes high frequency data to the telemetry circuitry 704 and blocks the high frequency data from the sensing circuitry 702. For example, the isolating filter circuit 706 receives high frequency RF communications data from the antenna 304 of the tail extension 104. The isolating filter circuit 706 passes the high frequency RF communications data to the telemetry circuit 704. The isolating filter circuit 706 blocks the RF communications data from the sensing circuitry 702.

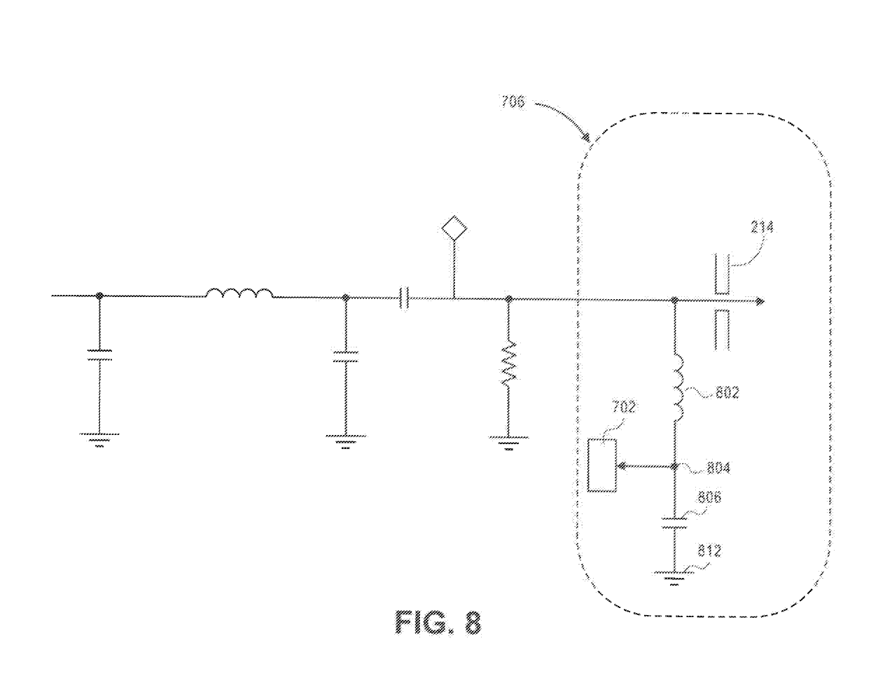

FIG. 8 provides a detailed illustration of the isolating filter circuit 706 of FIG. 7. The isolating filter circuit 706 includes an inductive element 802 and a capacitive element 806 that are connected in series with one another. An intermediate node 804 is positioned between the inductive element 802 and the capacitive element. The sensing circuitry 702 is conductively joined to the intermediate node. The capacitive element 806 is configured to form an open circuit when experiencing low frequency cardiac sensing signals from the sensing electrode 210 of the tail extension 104. For example, the open circuit enables the sensing signals to electrically flow towards the sensing circuitry 702. Alternatively, the capacitor 806 is configured to form a closed circuit when experiencing high frequency RF transmissions to and/or from the antenna. The closed circuit capacitive element 806 passes the high frequency RF transmissions to a ground 812 and blocks the high frequency RF transmissions from the sensing circuitry 702. Optionally, alternative circuitry may be used to filter the low frequency data from the high frequency data provided on a single-strand conductor and antenna.

Returning to FIG. 7, by way of example, the external device 108 may represent a bedside monitor installed in a patient's home and utilized to communicate with the ICM device 100 while the patient is at home, in bed or asleep. The external device 108 may be a programmer used in the clinic to interrogate the device, retrieve data and program detection criteria and other features. The external device 108 may be a device that can be coupled over a network (e.g. the Internet) to a remote monitoring service, medical network and the like. The external device 108 facilitates access by physicians to patient data as well as permitting the physician to review real-time ECG signals while being collected by the ICM device 100.

The microcontroller 714 is coupled to a memory 722 by a suitable data/address bus 726. The programmable operating parameters used by the microcontroller 714 are stored in memory 722 and used to customize the operation of the ICM device 100 to suit the needs of a particular patient. Such operating parameters define, for example, detection rate thresholds, sensitivity, automatic features, arrhythmia detection criteria, activity sensing or other physiological sensors, and electrode polarity, etc. The operating parameters of the ICM device 100 may be non-invasively programmed into the ICM device 100 through the telemetry circuitry 704. The telemetry circuitry 704 allows intracardiac electrograms and status information relating to the operation of the ICM device 100 to be sent to the external device 108 through an established communication link 724.

The ICM device 100 may further include magnet detection circuitry (not shown), coupled to the microcontroller 714, to detect when a magnet is placed over the unit. A magnet may be used by a clinician to perform various test functions of the ICM device 100 and/or to signal the microcontroller 714 that the external device 108 is in place to receive or transmit data to the microcontroller 714 through the telemetry circuitry 704.

The ICM device 100 can further include one or more physiologic sensor 728. Such sensors are commonly referred to (in the pacemaker arts) as “rate-responsive” or “exercise” sensors. The physiological sensor 728 may further be used to detect changes in the physiological condition of the heart, or diurnal changes in activity (e.g., detecting sleep and wake states). Signals generated by the physiological sensors 728 are passed to the microcontroller 714 for analysis and optional storage in the memory 722 in connection with the cardiac activity data, markers, episode information and the like. While shown as included within the ICM device 100, the physiologic sensor(s) 728 may be external to the ICM device 100, yet still be implanted within or carried by the patient. Examples of physiologic sensors might include sensors that, for example, activity, temperature, sense respiration rate, pH of blood, ventricular gradient, activity, position/posture, minute ventilation (MV), and so forth.

A battery 730 provides operating power to all of the components in the ICM device 100. The battery 730 is capable of operating at low current drains for long periods of time. The battery 730 also desirably has a predictable discharge characteristic so that elective replacement time can be detected. As one example, the ICM device 100 employs lithium/silver vanadium oxide batteries. The battery 730 may afford various periods of longevity (e.g. three years or more of device monitoring). In alternate embodiments, the batter 730 could be rechargeable. See for example, U.S. Pat. No. 7,294,108, Cardiac event microrecorder and method for implanting same, which is hereby incorporated by reference.

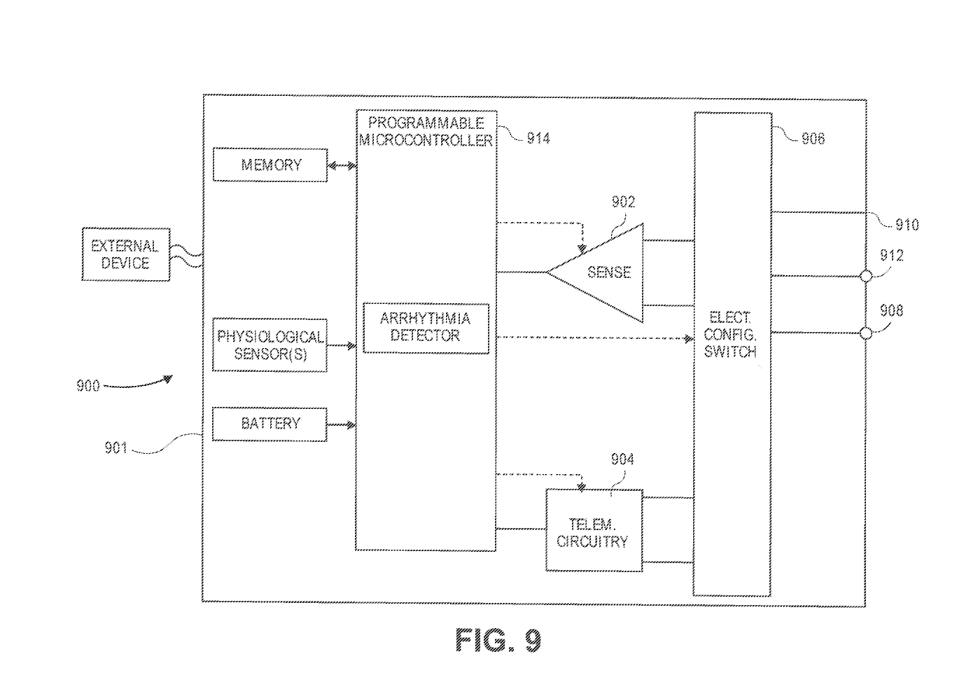

FIG. 9 illustrates a block-diagram of an alternative embodiment of the circuitry of an ICM device 900. The ICM device 900 has a device housing 901 to hold the electronic/computing components. The ICM device 900 is equipped with telemetry circuitry 904 and sensing circuitry 902. The telemetry circuitry 904 may include one or more transceivers. The sensing circuitry 902 is selectively coupled to one or more electrodes that perform sensing operations to detect cardiac activity data indicative of cardiac activity. The sensing circuitry 902 may include dedicated sense amplifiers, multiplexed amplifiers, or shared amplifiers. It may further employ one or more low power, precision amplifiers with programmable gain and/or automatic gain control, bandpass filtering, and threshold detection circuit to selectively sense the cardiac signal of interest.

In the present embodiment, the sensing electrode 210 positioned on the tail extension 104 is coupled to a terminal 908. The device housing 102 further includes a connector (not shown) with at least one terminal 910 and preferably a second terminal 912. The terminals 910, 912 may be coupled to additional sensing electrodes positioned on the device housing, on the tail extension, or located otherwise. For example, the terminals 910, 912 may be coupled to the sensing electrodes 212, 213, and/or 215 of FIG. 2. Additionally or alternatively, the terminals 910, 912 may be connected to one or more leads having one or more electrodes provided thereon, where the electrodes are located in various locations about the heart. The type and location of each electrode may vary.

A switch 906 is electrically coupled between the terminals 908, 910, 912 and the telemetry circuitry 904 and the sensing circuitry 902. In the present example, the switch 906 is provided to allow selection of different electrode configurations connected to the terminals 908, 910, and 912 under the control of a microcontroller 914. The switch 906 is controlled by a control signal from the microcontroller 914. The switch 906 is used to determine the sensing polarity of the cardiac signal by selectively closing the appropriate electrical circuitry paths. For example, the switch 906 comprises a low pass filter branch and a band pass filter branch (not shown). The low pass filter branch is configured to pass the low frequency cardiac sensed signals from the sensing electrode 210 coupled to the conductor 302 of the tail extension 104 to the sensing circuitry 902. The band pass filter branch is configured to pass the high frequency RF communications, in a frequency range that includes approximately 2.4 GHz, from the antenna 304 of the tail extension 104 to the telemetry circuitry 904.

A physician implants the ICM device 100 into a subcutaneously location in the patient via a tail implant tool. The tail implant tool comprises a lumen with a distal end and a proximal end. The lumen is sized and shaped to receive the tail extension 104 of the ICM device 100 within the lumen. The distal end 206 of tail extension 104 is inserted into the distal end of the lumen of the tail implant tool. The physician inserts the distal end of the lumen into a subcutaneous area of the patient located proximate to the ROI in the heart. For example, the physician may insert the distal end of the lumen of the tail implant tool into the patient in order to position the sensing electrode 210 of the tail extension 104 proximate one side of the heart. The physician may position the device housing 102 proximate an opposite side of the heart. Optionally, the physician may position the tail extension 104 and the device housing 102 on the same side of the heart. The tail implant tool releases the tail extension 104, and is removed from the patient while leaving the tail extension 104 of the ICM device 100 implanted in the patient. Optionally, alternative tools may be utilized to implant the ICM device 100 into the patient.

The various methods as illustrated in the FIGS. and described herein represent exemplary embodiments of methods. The methods may be implemented in software, hardware, or a combination thereof. In various of the methods, the order of the steps may be changed, and various elements may be added, reordered, combined, omitted, modified, etc. Various of the steps may be performed automatically (e.g., without being directly prompted by user input) and/or programmatically (e.g., according to program instructions).

Various modifications and changes may be made as would be obvious to a person skilled in the art having the benefit of this disclosure. It is intended to embrace all such modifications and changes and, accordingly, the above description is to be regarded in an illustrative rather than a restrictive sense.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

All references, including publications, patent applications and patents, cited herein are hereby incorporated by reference to the same extent as if each reference were individually and specifically indicated to be incorporated by reference and were set forth in its entirety herein.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f), unless and until such claim limitations expressly use the phrase “means for” followed by a statement of function void of further structure.

1. An implantable cardiac device, comprising:
a device housing having sensing circuit and radio frequency (RF) communications circuit housed within the housing; and
a tail extension having a proximal end, a distal end and an extension body extending there between, the proximal end coupled to the housing, the extension body formed of a flexible material and including a single conductor that includes a proximal end conductively coupled to the sensing and RF communications circuit;
at least a portion of the single conductor forming an antenna to be utilized by the RF communications circuit to communicate with an external device;
an electrode provided on the extension body and conductively coupled to the single conductor, the single conductor being adapted to transmit sensed cardiac signals from the electrode to the sensing circuit, the tail extension being adapted for implantation in a subcutaneous area with the electrode located proximate to a region of interest (ROI) of a heart; and
a filter circuit conductively coupled between the single conductor and the sensing and RF communications circuits, the filter circuit configured to block RF transmissions from reaching the sensing circuit.

2. The device of claim 1, wherein the portion of the at least one conductor that forms the antenna is coupled to the electrode.

3-4. (canceled)

5. The device of claim 1, wherein the proximal end of the tail extension is joined directly at a non-header interface on a surface of the housing.

6. The device of claim 1, further comprising a feed-through assembly joined to the device housing, the feed-through assembly including a single conductor extending there through, the conductor having a proximal end connected to the sensing and RF communications circuits and having a distal end projecting from the feed-through assembly.

7. (canceled)

8. The device of claim 1, wherein the filter circuit includes an inductive element and capacitive element connected in series with one another at an intermediate node, the sensing circuit joined to the intermediate node, the capacitor configured to form an open circuit when experiencing cardiac sensing signals and to form a closed circuit when experiencing RF transmissions.

9. The device of claim 1, wherein the filter circuit comprises a low pass filter branch configured to pass cardiac sensed signals and a band pass filter branch configured to pass RF communications in a frequency range that includes approximately 2.4 GHz.

10. The device of claim 1, wherein the extension body has a predetermined length between the distal and proximal ends, the predetermined length being tuned based on a center frequency of the RF communications bandwidth.

11. A method for implanting a cardiac device, the method comprising:
positioning a device subcutaneously, the device comprising a device housing having a sensing circuit and a radio frequency (RF) communications circuit housed within the housing, the device further comprising a tail extension having an extension body with a proximal end joined to the device housing, the extension body formed of a flexible material and including at least one conductor;
positioning the tail extension in a subcutaneous area with an electrode provided on the tail extension located proximate to a region of interest (ROI) in a heart;
collecting cardiac signals from the ROI utilizing the electrode provided on the tail extension; and
conveying RF communications data to an external device utilizing an antenna that is formed from at least a portion of the at least one conductor.

12. The method of claim 11, wherein the positioning operation includes locating a distal end of the tail extension remote from the device housing and an electrode provided at the distal end of the tail extension located proximate to a region of interest in a heart

13. The method of claim 11, wherein the at least one conductor includes first and second conductors, the first conductor coupled to the electrode and configured to carry the cardiac signals, the second conductor forming the antenna and configured to carry the RF communications data to or from the RF communications circuit.

14. The method of claim 11, further comprising filtering the cardiac signals to isolated the RF communications circuit from the cardiac signals sensed over the at least one conductor; and filter the RF communications to isolate the sensing circuit from the RF communications data carried by the at least one conductor.

15. The method of claim 11, wherein the collecting and conveying operations utilize a common conductor in the tail extension.

16. The method of claim 11, the filtering operation comprises low pass filtering to pass the cardiac signals along a sensing branch and band pass filtering to pass RF communications data that is in a frequency range that includes approximately 2.4 GHz, along a communications branch.

17. The method of claim 11, further comprising inserting the tail extension into a lumen in a tail implant tool, inserting the tail implant tool subcutaneously to a location proximate to the ROI, and removing the tail implant tool while leaving the tail extension implanted.

18. The method of claim 11, wherein the ROI is located proximate to an atrium of the heart such that the cardiac signals include sensed P-waves.

19. A method for providing an implantable cardiac device, the method comprising:
providing a device comprising a device housing having a sensing circuit and a radio frequency (RF) communications circuit housed within the housing;
joining a tail extension to the device housing, the tail extension having an extension body with a proximal end joined to the device housing, the extension body formed of a flexible material and including at least one conductor;
positioning an electrode on the tail extension to sense cardiac signals when the tail extension is located proximate to a region of interest (ROI) in a heart;
forming an antenna from at least a portion of the at least one conductor; and
tuning the antenna and RF communications circuit to convey RF communications data to an external device utilizing a predetermined communications frequency.

20. The method of claim 19, wherein the tuning operation includes tuning the antenna to utilize the predetermined communications frequency centered at one of 2.4 GHz and 400 MHz.

21. An implantable cardiac device, comprising:
a device housing having sensing circuit and radio frequency (RF) communications circuit housed within the housing; and
a tail extension having a proximal end, a distal end and an extension body extending there between, the extension body formed of a flexible material and including first and second conductors; and
an electrode provided on the extension body and conductively coupled to the first conductor and the sensing circuit, the tail extension being adapted for implantation in a subcutaneous area with the electrode located proximate to a region of interest (ROI) of a heart; and
a feed-through assembly coupled to the device housing and adapted to electrically couple the first conductor to the sensing circuit and the second conductor to the RF communications circuit, wherein the first conductor is coupled to the electrode and configured to carry cardiac sensed signals to the sensing circuit and wherein at least a portion of the second conductor forms the antenna and is configured to carry communications data to or from the RF communications circuit to communicate with an external device.
